(19) United States
(12) Patent Application Publication
(10) Pub. No.: US 2011/0195238 A1
(43) Pub. Date: Aug. 11, 2011

(54) ANTHRAPYRIDONE COLORING MATTER, SALT THEREOF, INK COMPOSITION AND COLORED BODY

(75) Inventors: Daisuke Ono, Tokyo (JP); Yutaka Ishii, Tokyo (JP); Noriko Kajiura, Tokyo (JP); Hiroyuki Matsumoto, Tokyo (JP)

(73) Assignee: NIPPON KAYAKU KABUSHIKI KAISHA, Tokyo (JP)

(21) Appl. No.: 13/125,036

(22) PCT Filed: Oct. 15, 2009
(86) PCT No.: PCT/JP2009/067835

§ 371 Date: Apr. 19, 2011

(30) Foreign Application Priority Data
Oct. 22, 2008 (JP) 2008-271641

(51) Int. Cl. B32B 3/10; C07D 221/18; C09D 11/02; B41J 2/175

(52) U.S. Cl. 428/195.1; 546/76; 106/31.77; 347/20; 347/85

(57) ABSTRACT

Disclosed is an ink composition containing a magenta dye which has a hue and definition suitable for inkjet recording and provides recorded matter having excellent fastness such as light resistance, ozone gas resistance and the like. Also disclosed is the magenta dye. The ink composition contains, as a dye, at least one anthrapyridone dye represented by formula (1) or a salt thereof. In formula (1), Xₐ-X_{c} each independently represents an anilino group substituted by a carboxy group, a hydroxy group, or the like; R represents a hydrogen atom or the like; R₁ represents an alkyl group or the like; and R₃ and R₄ each independently represents a hydrogen atom or the like.

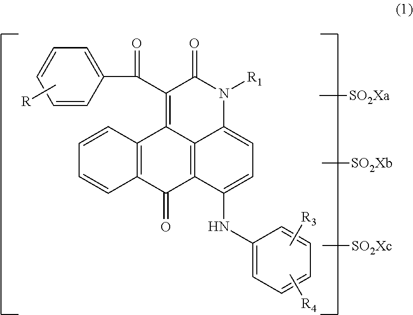

### TECHNICAL FIELD

The present invention relates to a novel anthrapyridone coloring matter or a salt thereof, a magenta ink composition containing an anthrapyridone coloring matter or a salt thereof, and a colored body obtained by coloring with the composition or the like.

### BACKGROUND ART

In connection with a recording method by an ink jet printer, which is one of typical methods among a variety of color recording methods, various types of discharge systems of the ink have been developed; however, any of them executes recording by generating ink droplets, which are attached to a variety of record-receiving materials (e.g., paper, film, and fabric, etc.). According to this method, a recording head is not brought into direct contact with the record-receiving material; therefore, generation of noise can be reduced thus achieving silent recording. In addition, due to having the features of a reduced size, an increased speed and coloring that can be readily achieved, prevalence in recent years has rapidly progressed, and great advancement hereafter is expected.

Aqueous inks containing a water soluble dye dissolved in an aqueous medium have been used as conventional inks of fountain pens, felt pens etc., and inks for ink jet recording. Furthermore, to these aqueous inks is generally added a water soluble organic solvent in order to prevent pen tips or ink discharge nozzles from clogging with the ink. For these conventional inks, demanded are abilities to generate a recorded image with satisfactory density, probability of avoiding occurrence of clogging at pen tips and nozzles, favorable drying characteristics on the record-receiving materials, suppression of bleeding, superior storage stability, and the like. In addition, formed images having various types of fastness properties such as water resistance, light resistance, moisture resistance, etc, are needed.

On the other hand, for recording image or character information displayed on a color display of a computer in full color by an ink jet printer, subtractive color mixing generally with four inks having different colors, yellow (Y), magenta (M), cyan (C) and black (K) has been employed for presentation. In order to reproduce an additive color mixing image formed with red (R), green (G), blue (B) on a CRT display and the like as accurately as possible using subtractive color mixing, it is desired that each Y, M and C are brilliant, and have a hue as approximate as possible to each standard.

Applications of ink jet printers have expanded from compact printers for OA (Office Automation) to large printers for industrial use, and thus various types of fastness properties such as water resistance, moisture resistance, light resistance and gas resistance have been desired more than ever.

Water resistance has been greatly improved by coating inorganic fine particles such as porous silica, a cationic polymer, an alumina sol or a special ceramic, capable of absorbing the coloring matter in an ink, on the surface of a record-receiving material together with a PVA resin or the like.

The moisture resistance referred to herein means resistance against a phenomenon of bleeding of a coloring matter in a record-receiving material when the record-receiving material colored is stored in a highly humid atmosphere. When there exists bleeding of the coloring matter, images that require photographic image quality with high definition, in particular, have markedly deteriorated image grade; therefore, minimizing such bleeding as far as possible is important.

A technique for significantly improving light resistance has not yet been established, and many of magenta coloring matters among coloring matters of four primary colors of Y, M, C and K particularly have originally inferior light resistance. Thus, improvement of the light resistance of magenta coloring matters has been an important issue. In addition, opportunities of printing photographs at home have increased along with recent popularization of digital cameras, and thus discoloration and fading of the image during storing the obtained recorded matter owing to an oxidizing gas in the air has been also problematic. The oxidizing gas reacts with the coloring matter on or in the record-receiving material, and allows the printed image to be discolorated and faded. Among the oxidizing gases, ozone gas has been considered as being the main causative substance that promotes the discoloration and fading phenomenon of ink jet recording images. Since this discoloration and fading phenomenon is characteristic in ink jet images, improvement of the ozone gas resistance is one of the most significant problems.

Typical examples of magenta coloring matters used in aqueous inks for ink jet recording include xanthene coloring matters and azo coloring matters prepared using an H acid. However, xanthene coloring matters are very inferior in light resistance although they are very superior in hue and brilliance. On the other hand, some of azo coloring matters prepared using an H acid are superior in hue and water resistance, they are inferior in light resistance, gas resistance, and brilliance. In this type, magenta coloring matters having superior brilliance and light resistance were also developed; however, as compared with coloring matters having other hue such as cyan coloring matters typified by copper phthalocyanine coloring matters, yellow coloring matters, etc., magenta coloring matters have a still lower level of light resistance.

Although magenta coloring matters having superior brilliance and light resistance are exemplified by anthrapyridone coloring matters (for example, see Patent Documents 1 to 12), any one that satisfies all requirements of hue, brilliance, light resistance, water resistance, gas resistance, and solution stability has not been obtained.
Patent Document 1: Japanese Unexamined Patent Application, Publication No. H10-306221 (pp. 1-3, and 7-18)
Patent Document 2: Japanese Unexamined Patent Application, Publication No. 2000-109464 (pp. 1-2, and 8-12)
Patent Document 3: Japanese Unexamined Patent Application, Publication No. 2000-169776 (pp. 1-2, and 6-9)
Patent Document 4: Japanese Unexamined Patent Application, Publication No. 2000-191660 (pp. 1-3, and 11-14)
Patent Document 5: Japanese Unexamined Patent Application, Publication No. 2000-256587 (pp. 1-3, and 7-18)
Patent Document 6: Japanese Unexamined Patent Application, Publication No. 2001-72884 (pp. 1-2, and 8-11)
Patent Document 7: Japanese Unexamined Patent Application, Publication No. 2001-139836 (pp. 1-2, and 7-12)
Patent Document 8: PCT International Publication No. 2004/104108 (pp. 20-36)
Patent Document 9: Japanese Unexamined Patent Application, Publication No. 2003-192930 (pp. 1-4, and 15-18)
Patent Document 10: Japanese Unexamined Patent Application, Publication No. 2005-8868 (pp. 1-3, and 15-22)
Patent Document 11: Japanese Unexamined Patent Application, Publication No. 2005-314514 (pp. 1-3, and 15-20)
Patent Document 12: PCT International Publication No. 2006/075706

### DISCLOSURE OF THE INVENTION

### Problems to be Solved by the Invention

An object of the present invention is to provide an ink composition containing a magenta coloring matter which has high solubility in water as well as a hue and brilliance suited for ink jet recording, and provides recorded matter having various types of superior fastness properties such as water resistance, moisture resistance and gas resistance, and also to provide such a coloring matter.

### Means for Solving the Problems

The present inventors thoroughly investigated in order to solve the foregoing problems, and consequently found that an anthrapyridone coloring matter represented by a certain formula solves the aforementioned problems. Thus, the present invention was completed.

Accordingly, a first aspect of the present invention provides an ink composition containing as a coloring matter at least one anthrapyridone coloring matter represented by the following formula (1) or a salt thereof

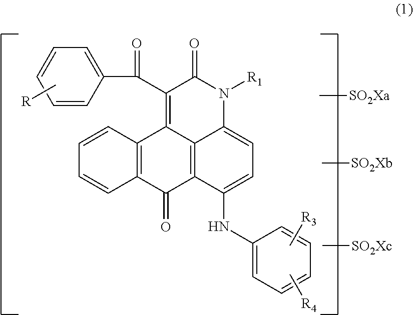

in the formula (1),

Xₐ to X_{c} each independently represent an anilino group substituted with a carboxy group, a mono- or di-alkylamino group substituted with a carboxy group, or a hydroxy group, and at least one of Xₐ to X_{c} represents a group other than a hydroxy group;

R represents a hydrogen atom, a sulfo group, a carboxy group, an alkoxy group, an alkylthio group, a carbamoyl group, a cyano group, an alkyl group, an anilino group, a phenoxy group, an amino group, a hydroxy group, or a mercapto group;

R₁ represents a hydrogen atom, an alkyl group, a hydroxyalkyl group, a phenyl group, a mono- or di-alkylaminoalkyl group, or a cyano alkyl group; and

R₃ and R₄ each independently represent a hydrogen atom or an alkyl group.

A second aspect of the present invention provides the ink composition according to the first aspect, in which the anthrapyridone coloring matter represented by the above formula (1) or a salt thereof is an anthrapyridone coloring matter represented by the following formula (2) or a salt thereof

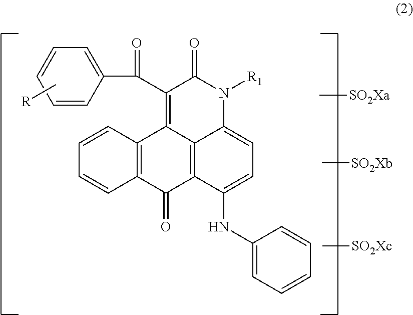

in the formula (2), Xₐ to X_{c}, R, and R₁ are as defined in the formula (1).

A third aspect of the present invention provides the ink composition according to the first aspect, in which the anthrapyridone coloring matter represented by the above formula (1) or a salt thereof is an anthrapyridone coloring matter represented by the following formula (3) or a salt thereof

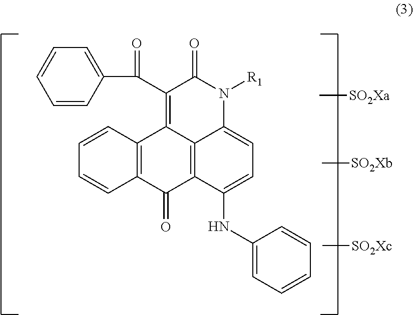

in the formula (3), Xₐ to X_{c}, and R₁ are as defined in the formula (1).

A fourth aspect of the present invention provides the ink composition according to the first aspect, in which the anthrapyridone coloring matter represented by the above formula (1) or a salt thereof is an anthrapyridone coloring matter represented by the following formula (4) or a salt thereof

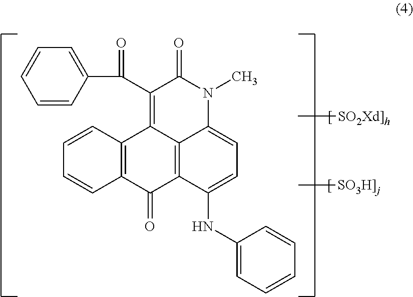

in the formula (4),

X_{d} represents an anilino group substituted with a carboxy group, or a mono- or di-alkylamino group substituted with a carboxy group; h and j are both an average value; h is from 1.6 to 2.5; j is from 0.5 to 1.4; and the sum of h and j is 3.0.

A fifth aspect of the present invention provides the ink composition according to the first aspect, in which the anthrapyridone coloring matter represented by the above formula (1) or a salt thereof is a coloring matter or a salt thereof obtained by reacting a compound represented by the following formula (5) with at least one amine selected from the group consisting of aniline substituted with a carboxy group, and a mono- or di-alkylamine substituted with a carboxy group

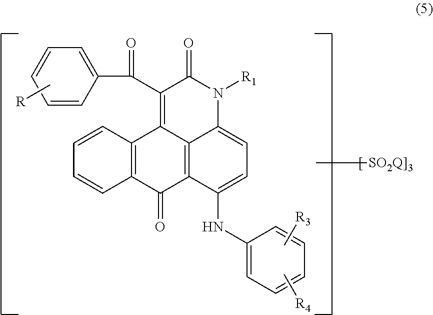

in the formula (5), Q represents a halogen atom; and R, R₁, R₃ and R₄ are as defined in the formula (1).

A sixth aspect of the present invention provides the ink composition according to any one of the first to fifth aspects containing water and a water soluble organic solvent.

A seventh aspect of the present invention provides the ink composition according to any one of the first to sixth aspects, in which the content of inorganic impurities in the total mass of the anthrapyridone coloring matter or a salt thereof according to any one of the first to fifth aspects contained in the ink composition as a coloring matter is no greater than 1% by mass.

An eighth aspect of the present invention provides the ink composition according to any one of the first to seventh aspects, in which the content of the anthrapyridone coloring matter according to any one of the first to fifth aspects contained in the ink composition as a coloring matter is 0.1 to 20% by mass relative to the total mass of the ink composition.

A ninth aspect of the present invention provides the ink composition according to any one of the first to eighth aspects, which is for use in ink jet recording.

A tenth aspect of the present invention provides an ink jet recording method including recording on a record-receiving material using as an ink the ink composition according to any one of the first to ninth aspects by discharging ink droplets of the ink in response to recording signals.

An eleventh aspect of the present invention provides the ink jet recording method according to the tenth aspect, wherein the record-receiving material is a communication sheet.

A twelfth aspect of the present invention provides the ink jet recording method according to the eleventh aspect, in which the communication sheet is a sheet having an ink-receiving layer containing a porous white inorganic substance.

A thirteenth aspect of the present invention provides a colored body which was colored with the ink composition according to any one of the first to eighth aspects.

A fourteenth aspect of the present invention provides the colored body according to the thirteenth aspect, in which the coloring was carried out with an ink jet printer.

A fifteenth aspect of the present invention provides an ink jet printer equipped with a vessel containing the ink composition according to any one of the first to eighth aspects.

A sixteenth aspect of the present invention provides an anthrapyridone coloring matter represented by the following formula (4) or a salt thereof

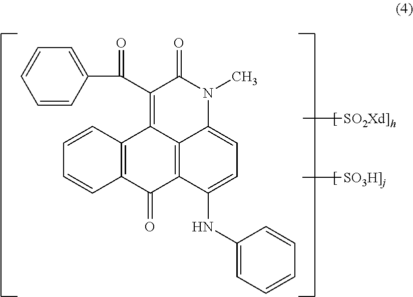

in the formula (4),

X_{d} represents an anilino group substituted with a carboxy group, or a mono- or di-alkylamino group substituted with a carboxy group; h and j are both an average value; h is from 1.6 to 2.5; j is from 0.5 to 1.4; and the sum of h and j is 3.0.

### Effects of the Invention

The anthrapyridone coloring matter represented by the above formula (1) or a salt thereof of the present invention exhibits a hue of very high brilliance and brightness on ink jet recording papers, and has a characteristic feature of favorable filterability in the step of producing an ink composition on membrane filters. In addition, the ink composition of the present invention containing the coloring matter or a salt thereof exhibits favorable storage stability, without being accompanied by solid deposition, physical property alteration, color change and the like after storage for a long period of time. Moreover, printed matter obtained using the anthrapyridone coloring matter or a salt thereof of the present invention as a magenta ink for ink jet recording exhibits an ideal magenta hue without need of selection of the record-receiving material (paper, film, etc.). Additionally, the magenta ink composition of the present invention also enables the hue of photographic color images to be strictly reproduced on papers. Furthermore, even if recording is carried out on a record-receiving material including inorganic fine particles coated on its surface, such as an exclusive ink jet paper (or film) for photo image quality, favorable various fastness properties, i.e., ozone resistance, water resistance, moisture resistance and the like, as well as superior long-term storage stability of recorded image can be achieved. Accordingly, the anthrapyridone coloring matter represented by the above formula (1) or a salt thereof, and the ink composition containing the same are very useful for use in ink jet recording.

### PREFERRED MODE FOR CARRYING OUT THE INVENTION

The present invention is explained in detail. The anthrapyridone coloring matter or a salt thereof of the present invention is a magenta coloring matter, and substantially a mixture. In addition, for the sake of simplicity, both the anthrapyridone coloring matter and a salt thereof of the present invention are inclusively referred to as “the anthrapyridone coloring matter of the present invention” briefly herein below. Unless otherwise stated in particular herein, functional groups such as sulfo groups and carboxy groups are represented in the form of their free acids.

The coloring matter of the present invention is represented by the above formula (1).

In the above formula (1), the anilino group substituted with a carboxy group in Xₐ to X_{c} may be substituted with usually 1 to 4, preferably 1 to 3, more preferably 1 or 2 carboxy group(s), and still more preferably 1 carboxy group.

Specific examples include those substituted with one carboxy group such as 2-carboxyanilino, 3-carboxyanilino and 4-carboxyanilino; those substituted with two carboxy groups such as 2,5-dicarboxyanilino and 3,5-dicarboxyanilino; and the like.

Although the position of substitution with a carboxy group is not particularly limited, provided that the position of substitution with an amino group is position 1, when substituted with one carboxy group, it is preferably substituted at position 2. Similarly, when substituted with two carboxy groups, they are preferably substituted at positions 2 and 5, or positions 3 and 5, and more preferably substituted at positions 3 and 5.

The mono- or di-alkylamino group substituted with a carboxy group in the Xₐ to X_{c} is exemplified by a straight, branched, or cyclic C1-C10, preferably C1-C6, and more preferably C1-C4 mono- or di-alkylamino group. The monoalkylamino group is preferably straight, and the dialkylamino group is preferably straight or cyclic.

Specific examples of the monoalkylamino group include those substituted with one carboxy group such as carboxymethylamino, 2-carboxyethylamino, 3-carboxypropylamino and 5-carboxypentylamino; those substituted with two carboxy groups such as 1,2-dicarboxyethylamino and 1,3-dicarboxypropylamino; and the like.

The monoalkylamino group is preferably a mono(carboxy-substituted straight C1-C4 alkyl)amino group, and more preferably carboxymethylamino.

Preferable examples of the straight dialkylamino group include di(carboxy-substituted straight C1-C4 alkyl)amino groups such as bis(carboxymethyl)amino, and the like.

Examples of the cyclic dialkylamino group include 5- or 6-membered cyclic nitrogen-containing aliphatic heterocyclic groups including 1 or 2 nitrogen atoms, preferably 1 nitrogen atom as an atom constructing the ring, such as piperidine and pyrrolidine; carboxy-substituted 5- or 6-membered nitrogen-containing aliphatic heterocyclic groups such as 4-carboxypiperidine (isonipecotic acid) and 2-carboxypiperidine (proline); and the like. These heterocyclic groups bind to a sulfur atom in the formula (1) via the nitrogen atom included as an atom constructing the ring.

The dialkylamino group in Xₐ to X_{c} is more preferably a bis(carboxy-substituted straight C1-C4 alkyl)amino group, or a carboxy-substituted 5- or 6-membered cyclic nitrogen-containing aliphatic heterocyclic group.

Although the number of substituent is not limited, the number is usually 1 to 4, preferably 1 to 3, more preferably 1 or 2, and still more preferably 1.

Of the Xₐ to X_{c}, at least one is preferably an anilino group substituted with a carboxy group, and it is more preferred that at least one is an anilino group substituted with a carboxy group, and the rest is a hydroxy group.

In the above formula (1), exemplary alkoxy group represented by R may have a straight or branched chain, and preferably has a straight chain. With respect to the range of the number of carbon atoms, usually C1-C8, preferably C1-C6, and more preferably C1-C4 are exemplified.

Specific examples of the alkoxy group include those having a straight chain such as methoxy, ethoxy, n-propoxy, n-butoxy, n-pentoxy, n-hexyloxy, n-heptyloxy and n-octyloxy; those having a branched chain such as isopropoxy, isobutoxy, sec-butoxy and t-butoxy; and the like.

The alkylthio group represented by R in the above formula (1) may have a straight or branched chain, and preferably has a straight chain. With respect to the range of the number of carbon atoms, usually C1-C6, and preferably C1-C4 are exemplified.

Specific examples of the alkylthio group include those having a straight chain such as methylthio, ethylthio, n-propylthio, n-butylthio, n-pentylthio and n-hexylthio; those having a branched chain such as isopropylthio, isobutylthio, sec-butylthio and t-butylthio; and the like.

The alkyl group represented by R in the above formula (1) may have a straight, branched chain or a cyclic structure, and has preferably a straight or branched chain and more preferably a straight chain. With respect to the range of the number of carbon atoms, usually C1-C8, preferably C1-C6, and more preferably C1-C4 are exemplified.

Specific examples of the alkyl group include those having a straight chain such as methyl, ethyl, n-propyl, n-butyl, n-pentyl, n-hexyl, n-heptyl and n-octyl; those having a branched chain such as isopropyl, isobutyl, sec-butyl and t-butyl; those having a cyclic structure such as cyclopropyl, cyclobutyl, cyclopentyl or cyclohexyl; and the like.

The R represents preferably a hydrogen atom, an alkoxy group, or an alkyl group, more preferably a hydrogen atom, a C1-C4 alkoxy group, or a C1-C4 alkyl group, and still more preferably a hydrogen atom.

The alkyl group represented by R₁ in the above formula (1) may have a straight, branched chain, or a cyclic structure, preferably a straight or branched chain, and more preferably a straight chain. Those having a straight or branched chain, and the range of the number of carbon atoms are as defined for the alkyl group in connection with R described above, including preferable options thereof. Examples of the cyclic alkyl include cyclopropyl, cyclobutyl, cyclopentyl, cyclohexyl and the like, and cyclohexyl is particularly preferred.

The hydroxyalkyl group represented by R₁ in the above formula (1) is exemplified by hydroxy C1-C4 alkyl groups such as hydroxymethyl, 2-hydroxyethyl, 3-hydroxypropyl and 4-hydroxybutyl.

The mono- or di-alkylamino alkyl group represented by R₁ in the above formula (1) is exemplified by mono- or di-C1-C4 alkylamino C1-C4 alkyl groups such as dimethylaminomethyl, 2-dimethylaminoethyl and 2-diethylaminoethyl.

The cyano alkyl group represented by R₁ in the above formula (1) is exemplified by cyano C1-C4 alkyl groups such as cyanomethyl, 2-cyanoethyl, 3-cyanopropyl and 4-cyanobutyl.

The aforementioned R₁ is preferably a hydrogen atom or an alkyl group, more preferably a C1-C4 alkyl group, and still more preferably methyl.

The alkyl group represented by R₃ and R₄ in the above formula (1) may include a straight or branched chain, and straight groups are preferred. With respect to the range of the number of carbon atoms, usually C1-C8, preferably C1-C6, and more preferably C1-C4 are exemplified.

Specific examples of the alkyl group include those having a straight chain such as methyl, ethyl, n-propyl, n-butyl, n-pentyl, n-hexyl, n-heptyl and n-octyl; those having a branched chain such as isopropyl, isobutyl, sec-butyl, t-butyl and 2-ethylhexyl; and the like.

It is preferred that both R₃ and R₄ are a hydrogen atom.

The coloring matter represented by the above formula (1) is preferably a coloring matter represented by the above formula (2), and more preferably a coloring matter represented by the above formula (3).

In the above formulae (2) and (3), Xₐ to X_{c}, R, and R₁ which may be optionally selected are as defined in connection with the above formula (1) including preferable options, etc.

The coloring matter represented by the above formula (1) is particularly preferably a coloring matter represented by the above formula (4).

The anilino group substituted with a carboxy group; and the mono- or di-alkylamino group substituted with a carboxy group in the aforementioned X_{d} are as defined in connection with Xₐ to X_{c} in the above formula (1) including preferable options, etc. In particular, X_{d} is preferably an anilino group substituted with a carboxy group.

In the formula (4), h and j are an average value indicating the number of substitution with the substituted sulfamoyl groups and sulfo groups, respectively, each having X_{d}, and the sum of h and j is 3.0.

Here, h is usually from 1.6 to 2.5, preferably from 1.7 to 2.5, and more preferably from 1.8 to 2.4.

Also, j is usually from 0.5 to 1.4, preferably from 0.5 to 1.3, and more preferably from 0.6 to 1.2.

The coloring matter represented by the formula (4) of the present invention is substantially a mixture of coloring matters having an anthrapyridone structure substituted with three in total of a group represented by “—SO₂X_{d}”, and a sulfo group represented by “—SO₃H”. Therefore, values of h and j in the formula (4) may be calculated by carrying out HPLC analysis of the coloring matter mixture, and measuring the area ratio on the HPLC of each single coloring matter constituting the coloring matter mixture. By way of an example, a method of calculating h in a coloring matter mixture (A) having the following constitution is described below.

Results of HPLC analysis of dye mixture (A):

| Number of substitution with “—SO₂X_{d}” | HPLC area ratio (%) |
|---|---|
| 0 | A₁ |
| 1 | A₂ |
| 2 | A₃ |
| 3 | A₄ |

Method of calculating average value h in dye mixture (A)

h=[(0×A₁)+(1×A₂)+(2×A₃)+(3×A₄)]/(A₁+A₂+A₃+A₄)

The area ratio on the HPLC presented herein is a value derived by rounding the calculated h to one decimal place, using the found value up to one digit after the decimal point. It is to be noted that the average value j may be calculated similarly to the aforementioned calculation method of h, but may be conveniently calculated according to the formula of “j 3.0−h”.

The number of substitution with “—SO₂X_{d}” in the aforementioned HPLC analysis may be readily determined based on a mass derived by, for example, fractionating the peak of each single dye detected on the HPLC and subjecting the fraction to an instrumental analysis such as mass spectrometry. In brief, mass spectrometry is carried out in conjunction with measurement of LC as in LC/MS, and the number of substitution may be determined from thus derived mass.

The content of the coloring matter represented by the above formula (1) in the total mass of the coloring matter contained in the ink composition of the present invention is usually 75% to 100%, preferably 80% to 100%, and more preferably 85% to 100% all on mass basis.

In connection with Xₐ to X_{c}, R, R₁, R₃, R₄, X_{d}, h, and j optionally selected for the coloring matter each represented by the above formulae (1) to (4), combinations of preferable options are more preferred, and combinations of more preferable options are even more preferred. The same is applied to combinations of preferable options with more preferable options, etc.

The salt of the coloring matter represented by the above formula (1) is an inorganic or organic cation salt. Specific examples of the inorganic salt include alkali metal salts, alkaline earth metal salts and ammonium salts, and preferable inorganic salts include lithium, sodium and potassium salts, and ammonium salts. Furthermore, examples of the organic cation salt include salts with quaternary ammonium represented by the following formula (6) but not limited thereto. Also, free acids, and various types of salts thereof may constitute a mixture. For example, any combination such as a mixture of a sodium salt and an ammonium salt, a mixture of a free acid and a sodium salt, a mixture of a lithium salt, a sodium salt and an ammonium salt, and the like may be used. Physical property values such as solubility may vary corresponding to the type of the salt, and a mixture having physical properties suited for the object can be obtained by: selecting the type of the salt appropriately as needed; changing the ratio of the salts when a plurality of salts, etc., are included; or the like.

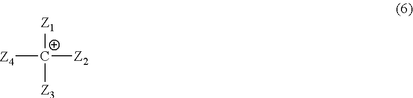

In the formula (6), Z₁ to Z₄ each independently represent a hydrogen atom, an alkyl group, a hydroxyalkyl group, or a hydroxyalkoxyalkyl group, and at least one is a group other than a hydrogen atom.

With regard to Z₁ to Z₄ in the quaternary ammonium represented by the formula (6), examples of the alkyl group include methyl, ethyl and the like; examples of the hydroxyalkyl group include hydroxymethyl, 2-hydroxyethyl, 3-hydroxypropyl, 2-hydroxypropyl, 4-hydroxybutyl, 3-hydroxybutyl, 2-hydroxybutyl and the like; and examples of the hydroxyalkoxyalkyl group include hydroxyethoxymethyl, 2-hydroxyethoxyethyl, 3-hydroxyethoxypropyl, 3-hydroxyethoxybutyl, 2-hydroxyethoxybutyl and the like.

Preferable salts of the coloring matter represented by the above formula (1) include each salt of sodium, potassium, lithium, monoethanolamine, diethanolamine, triethanolamine, monoisopropanolamine, diisopropanolamine, and triisopropanolamine, as well as an ammonium salt, and the like. More preferable salts are lithium, ammonium, and sodium salts.

In order to obtain a salt of the coloring matter represented by the above formula (1), sodium chloride is added to, for example, a reaction liquid containing the coloring matter represented by the above formula (1), or a solution of the wet cake or the dried matter of the coloring matter dissolved in water, and then salting-out and filtration of the mixture may be carried out. Thus, a sodium salt of the coloring matter can be obtained as a wet cake. In addition, after dissolving the wet cake in water again, hydrochloric acid is added to the mixture to adjust the pH to 1 to 2. The free acid or a mixture of the free acid and the sodium salt can be obtained by filtering thus resulting solid. Moreover, when potassium hydroxide, lithium hydroxide, aqueous ammonia, or quaternary ammonium represented by the formula (6) (usually, a quaternary ammonium salt having a halogen ion or the like as a counter anion), for example, is added to a free acid of the coloring matter represented by the formula (1) or a wet cake thereof while stirring with water to make alkali, each corresponding potassium salt, lithium salt, ammonium salt, or a quaternary ammonium salt represented by the above formula (6) is obtained.

Specific examples of the anthrapyridone coloring matter represented by the above formula (1) contained in the ink composition of the present invention are shown in Table 1 below, but not particularly limited thereto.

TABLE 1

| No. | Xa | Xb | Xc | R | R1 | R3 | R4 |
|---|---|---|---|---|---|---|---|
| 1 | OH | OH | NHCH2COOH | H | methyl | H | H |
| 2 | OH | NHCH2COOH | NHCH2COOH | H | methyl | H | H |
| 3 | NHCH2COOH | NHCH2COOH | NHCH2COOH | H | methyl | H | H |
| 4 | OH | OH | N(CH2COOH)2 | H | methyl | H | H |
| 5 | OH | N(CH2COOH)2 | N(CH2COOH)2 | H | methyl | H | H |
| 6 | N(CH2COOH)2 | N(CH2COOH)2 | N(CH2COOH)2 | H | methyl | H | H |
| 7 | OH | OH | 2-carboxy- | H | methyl | H | H |
| | | | phenylamino | | | | |
| 8 | OH | 2-carboxy- | 2-carboxy- | H | methyl | H | H |
| | | phenylamino | phenylamino | | | | |
| 9 | 2-carboxy- | 2-carboxy- | 2-carboxy- | H | methyl | H | H |
| | phenylamino | phenylamino | phenylamino | | | | |
| 10 | OH | OH | 3-carboxy- | H | methyl | H | H |
| | | | phenylamino | | | | |
| 11 | OH | 3-carboxy- | 3-carboxy- | H | methyl | H | H |
| | | phenylamino | phenylamino | | | | |
| 12 | 3-carboxy- | 3-carboxy- | 3-carboxy- | H | methyl | H | H |
| | phenylamino | phenylamino | phenylamino | | | | |
| 13 | OH | OH | 4-carboxy- | H | methyl | H | H |
| | | | phenylamino | | | | |
| 14 | OH | 4-carboxy- | 4-carboxy- | H | methyl | H | H |
| | | phenylamino | phenylamino | | | | |
| 15 | 4-carboxy- | 4-carboxy- | 4-carboxy- | H | methyl | H | H |
| | phenylamino | phenylamino | phenylamino | | | | |
| 16 | OH | OH | 4-carboxy- | H | methyl | H | H |
| | | | piperidin-1-yl | | | | |
| 17 | OH | 4-carboxy- | 4-carboxy- | H | methyl | H | H |
| | | piperidin-1-yl | piperidin-1-yl | | | | |
| 18 | 4-carboxy- | 4-carboxy- | 4-carboxy- | H | methyl | H | H |
| | piperidin-1-yl | piperidin-1-yl | piperidin-1-yl | | | | |
| 19 | OH | OH | NHCH2COOH | H | H | H | H |
| 20 | OH | NHCH2COOH | NHCH2COOH | H | H | H | H |
| 21 | NHCH2COOH | NHCH2COOH | NHCH2COOH | H | H | H | H |
| 22 | OH | OH | 2-carboxy- | H | H | H | H |
| | | | phenylamino | | | | |
| 23 | OH | 2-carboxy- | 2-carboxy- | H | H | H | H |
| | | phenylamino | phenylamino | | | | |
| 24 | 2-carboxy- | 2-carboxy- | 2-carboxy- | H | H | H | H |
| | phenylamino | phenylamino | phenylamino | | | | |
| 25 | OH | OH | NHCH2COOH | H | methyl | H | 3-methyl |
| 26 | OH | NHCH2COOH | NHCH2COOH | H | methyl | H | 3-methyl |
| 27 | NHCH2COOH | NHCH2COOH | NHCH2COOH | H | methyl | H | 3-methyl |
| 28 | OH | OH | 2-carboxy- | H | methyl | H | 3-methyl |
| | | | phenylamino | | | | |
| 29 | OH | 2-carboxy- | 2-carboxy- | H | methyl | H | 3-methyl |
| | | phenylamino | phenylamino | | | | |
| 30 | 2-carboxy- | 2-carboxy- | 2-carboxy- | H | methyl | H | 3-methyl |
| | phenylamino | phenylamino | phenylamino | | | | |
| 31 | OH | OH | NHCH2COOH | 4-methoxy | methyl | H | H |
| 32 | OH | NHCH2COOH | NHCH2COOH | 4-methoxy | methyl | H | H |
| 33 | NHCH2COOH | NHCH2COOH | NHCH2COOH | 4-methoxy | methyl | H | H |
| 34 | OH | OH | N(CH2COOH)2 | 4-methoxy | methyl | H | H |
| 35 | OH | N(CH2COOH)2 | N(CH2COOH)2 | 4-methoxy | methyl | H | H |
| 36 | N(CH2COOH)2 | N(CH2COOH)2 | N(CH2COOH)2 | 4-methoxy | methyl | H | H |
| 37 | OH | OH | 2-carboxy- | 4-methoxy | methyl | H | H |
| | | | phenylamino | | | | |
| 38 | OH | 2-carboxy- | 2-carboxy- | 4-methoxy | methyl | H | H |
| | | phenylamino | phenylamino | | | | |
| 39 | 2-carboxy- | 2-carboxy- | 2-carboxy- | 4-methoxy | methyl | H | H |
| | phenylamino | phenylamino | phenylamino | | | | |
| 40 | OH | OH | NHCH2COOH | H | H | H | 3-methyl |
| 41 | OH | NHCH2COOH | NHCH2COOH | H | H | H | 3-methyl |
| 42 | NHCH2COOH | NHCH2COOH | NHCH2COOH | H | H | H | 3-methyl |
| 43 | OH | OH | 2-carboxy- | H | H | H | 3-methyl |
| | | | phenylamino | | | | |
| 44 | OH | 2-carboxy- | 2-carboxy- | H | H | H | 3-methyl |
| | | phenylamino | phenylamino | | | | |
| 45 | 2-carboxy- | 2-carboxy- | 2-carboxy- | H | H | H | 3-methyl |
| | phenylamino | phenylamino | phenylamino | | | | |
| 46 | OH | OH | 3-carboxy- | H | H | H | 3-methyl |
| | | | phenylamino | | | | |
| 47 | OH | 3-carboxy- | 3-carboxy- | H | H | H | 3-methyl |
| | | phenylamino | phenylamino | | | | |
| 48 | 3-carboxy- | 3-carboxy- | 3-carboxy- | H | H | H | 3-methyl |
| | phenylamino | phenylamino | phenylamino | | | | |
| 49 | OH | OH | NHCH2COOH | H | methyl | 2-methyl | 5-methyl |
| 50 | OH | NHCH2COOH | NHCH2COOH | H | methyl | 2-methyl | 5-methyl |
| 51 | NHCH2COOH | NHCH2COOH | NHCH2COOH | H | methyl | 2-methyl | 5-methyl |
| 52 | OH | OH | 2-carboxy- | H | methyl | 2-methyl | 5-methyl |
| | | | phenylamino | | | | |
| 53 | OH | 2-carboxy- | 2-carboxy- | H | methyl | 2-methyl | 5-methyl |
| | | phenylamino | phenylamino | | | | |
| 54 | 2-carboxy- | 2-carboxy- | 2-carboxy- | H | methyl | 2-methyl | 5-methyl |
| | phenylamino | phenylamino | phenylamino | | | | |
| 55 | OH | OH | NHCH2COOH | H | methyl | H | 2-ethyl |
| 56 | OH | NHCH2COOH | NHCH2COOH | H | methyl | H | 2-ethyl |
| 57 | NHCH2COOH | NHCH2COOH | NHCH2COOH | H | methyl | H | 2-ethyl |
| 58 | OH | OH | 2-carboxy- | H | methyl | H | 2-ethyl |
| | | | phenylamino | | | | |
| 59 | OH | 2-carboxy- | 2-carboxy- | H | methyl | H | 2-ethyl |
| | | phenylamino | phenylamino | | | | |
| 60 | 2-carboxy- | 2-carboxy- | 2-carboxy- | H | methyl | H | 2-ethyl |
| | phenylamino | phenylamino | phenylamino | | | | |
| 61 | OH | OH | NHCH2COOH | H | methyl | H | 3-ethyl |
| 62 | OH | NHCH2COOH | NHCH2COOH | H | methyl | H | 3-ethyl |
| 63 | NHCH2COOH | NHCH2COOH | NHCH2COOH | H | methyl | H | 3-ethyl |
| 64 | OH | OH | 2-carboxy- | H | methyl | H | 3-ethyl |
| | | | phenylamino | | | | |
| 65 | OH | 2-carboxy- | 2-carboxy- | H | methyl | H | 3-ethyl |
| | | phenylamino | phenylamino | | | | |
| 66 | 2-carboxy- | 2-carboxy- | 2-carboxy- | H | methyl | H | 3-ethyl |
| | phenylamino | phenylamino | phenylamino | | | | |
| 67 | OH | OH | NHCH2COOH | H | H | H | 3-ethyl |
| 68 | OH | NHCH2COOH | NHCH2COOH | H | H | H | 3-ethyl |
| 69 | NHCH2COOH | NHCH2COOH | NHCH2COOH | H | H | H | 3-ethyl |
| 70 | OH | OH | 2-carboxy- | H | H | H | 3-ethyl |
| | | | phenylamino | | | | |
| 71 | OH | 2-carboxy- | 2-carboxy- | H | H | H | 3-ethyl |
| | | phenylamino | phenylamino | | | | |
| 72 | 2-carboxy- | 2-carboxy- | 2-carboxy- | H | H | H | 3-ethyl |
| | phenylamino | phenylamino | phenylamino | | | | |
| 73 | OH | OH | NHCH2COOH | 4-methyl | methyl | H | H |
| 74 | OH | NHCH2COOH | NHCH2COOH | 4-methyl | methyl | H | H |
| 75 | NHCH2COOH | NHCH2COOH | NHCH2COOH | 4-methyl | methyl | H | H |
| 76 | OH | OH | 2-carboxy- | 4-methyl | methyl | H | H |
| | | | phenylamino | | | | |
| 77 | OH | 2-carboxy- | 2-carboxy- | 4-methyl | methyl | H | H |
| | | phenylamino | phenylamino | | | | |
| 78 | 2-carboxy- | 2-carboxy- | 2-carboxy- | 4-methyl | methyl | H | H |
| | phenylamino | phenylamino | phenylamino | | | | |
| 79 | OH | OH | NHCH2COOH | 4-methyl | methyl | H | 3-methyl |
| 80 | OH | NHCH2COOH | NHCH2COOH | 4-methyl | methyl | H | 3-methyl |
| 81 | NHCH2COOH | NHCH2COOH | NHCH2COOH | 4-methyl | methyl | H | 3-methyl |
| 82 | OH | OH | 2-carboxy- | 4-methyl | methyl | H | 3-methyl |
| | | | phenylamino | | | | |
| 83 | OH | 2-carboxy- | 2-carboxy- | 4-methyl | methyl | H | 3-methyl |
| | | phenylamino | phenylamino | | | | |
| 84 | 2-carboxy- | 2-carboxy- | 2-carboxy- | 4-methyl | methyl | H | 3-methyl |
| | phenylamino | phenylamino | phenylamino | | | | |
| 85 | OH | OH | NHCH2COOH | 2-methyl | methyl | H | H |
| 86 | OH | NHCH2COOH | NHCH2COOH | 2-methyl | methyl | H | H |
| 87 | NHCH2COOH | NHCH2COOH | NHCH2COOH | 2-methyl | methyl | H | H |
| 88 | OH | OH | 2-carboxy- | 2-methyl | methyl | H | H |
| | | | phenylamino | | | | |
| 89 | OH | 2-carboxy- | 2-carboxy- | 2-methyl | methyl | H | H |
| | | phenylamino | phenylamino | | | | |
| 90 | 2-carboxy- | 2-carboxy- | 2-carboxy- | 2-methyl | methyl | H | H |
| | phenylamino | phenylamino | phenylamino | | | | |
| 91 | OH | OH | NHCH2COOH | 2-methyl | methyl | H | 3-methyl |
| 92 | OH | NHCH2COOH | NHCH2COOH | 2-methyl | methyl | H | 3-methyl |
| 93 | NHCH2COOH | NHCH2COOH | NHCH2COOH | 2-methyl | methyl | H | 3-methyl |
| 94 | OH | OH | 2-carboxy- | 2-methyl | methyl | H | 3-methyl |
| | | | phenylamino | | | | |
| 95 | OH | 2-carboxy- | 2-carboxy- | 2-methyl | methyl | H | 3-methyl |
| | | phenylamino | phenylamino | | | | |
| 96 | 2-carboxy- | 2-carboxy- | 2-carboxy- | 2-methyl | methyl | H | 3-methyl |
| | phenylamino | phenylamino | phenylamino | | | | |

The anthrapyridone coloring matter of the present invention is produced by, for example the following method. In the following formulae (9) to (11), any of Xₐ to X_{c}, R₁, R₃, R₄, and R which may be optionally selected is as defined in connection with the above formula (1).

Specifically, a reaction is carried out with 1 mol of an anthraquinone compound represented by the following formula (9) obtained in accordance with a well-known method as disclosed in Japanese Examined Patent Application, Publication No. H7-45629 and the like, and a benzoyl acetate ester having R as a substituent in the presence of a base such as sodium carbonate, potassium carbonate, sodium acetate or potassium acetate as a catalyst at 100 to 200° C. for 3 to 30 hrs in a solvent such as orthodichlorobenzene, monochlorobenzene, nitrobenzene or xylene. After completing the reaction, the mixture is cooled, and diluted with a C1-C4 alcohol such as methanol, ethanol or propanol. Thus precipitated solid is separated by filtration, followed by washing with the C1-C4 alcohol as needed and further washing with water or warm water, and then dried to obtain a compound represented by the following formula (10).

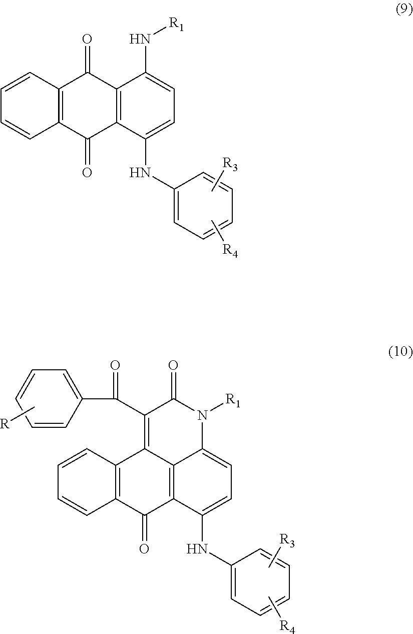

Thus obtained compound represented by the above formula (10) is subjected to chlorosulfonylation in chlorosulfonic acid at 40 to 120° C., and then at 70 to 80° C. after adding thionyl chloride to obtain a compound represented by the following formula (5).

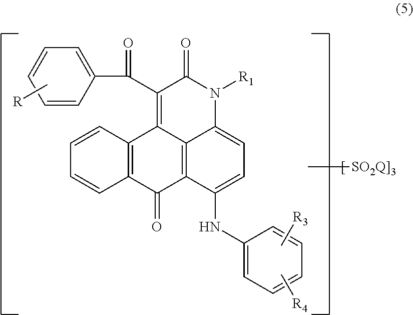

In the formula (5), Q represents a halogen atom, preferably a chlorine atom, a bromine atom, or an iodine atom, and more preferably a chlorine atom.

A base is used for adjusting the pH of a mixture of thus obtained compound represented by the formula (5), and at least one amine selected from the group consisting of aniline substituted with a carboxy group, and a mono- or di-alkylamine substituted with a carboxy group. Then the mixture is allowed to react with stirring, at room temperature or while cooling if necessary to obtain the coloring matter represented by the above formula (1) of the present invention. The aforementioned amine is preferably aniline substituted with a carboxy group.

It is to be noted that the compound represented by the above formula (5) is decomposed due to heat or the like, and thus a by-product in which Q is hydroxy is generated during the reaction with the amine selected from the aforementioned group. Since a high content of this by-product may be responsible for impairment of the effects of the invention, the content of the by-product may be usually no greater than 10%, preferably no greater than 8%, and more preferably no greater than 5% on the basis of the area ratio on HPLC relative to the sum total of the area ratio of the by-product and the coloring matter of the present invention in an HPLC analysis. The lower limit may be at least the detection limit of the analytical instrument, i.e., 0%.

In the coloring matter represented by the above formula (1) contained in the ink composition of the present invention, as the ratio of a group other than a hydroxy group in Xₐ to X_{c} increases, solubility of the dye in water decreases although fastness properties of the image recorded using the ink composition are improved. The ink composition of the present invention may not substantially contain water; however, it is preferable to contain water, i.e., the composition is preferably a water-based ink composition.

Therefore, taking into consideration the storage stability as the water-based ink composition, and fastness properties of the recorded image, for the purpose of improving solubility in water along with setting of the ratio of the group other than a hydroxy group in Xₐ to X_{c}, it is preferred to prepare an ink composition containing a coloring matter mixture of one or more types, preferably one to four types, and more preferably one to three types of the coloring matter represented by the above formula (1) of the present invention.

The coloring matter of the present invention is suited for dyeing natural and synthetic fiber materials or blends as a magenta coloring matter. Furthermore, such coloring matters are suited for the production of inks for writing and ink compositions for ink jet recording.

When the coloring matter represented by the above formula (1) is used as a coloring matter to be included in an ink composition, the content of inorganic substances such as chlorides of metal cations (for example, sodium chloride); and sulfuric acid salts of metal cations (for example, sodium sulfate); i.e., “inorganic impurities”, included in the total amount of the coloring matter is preferably as low as possible. The content of the inorganic impurities that may be acceptable is, for example, about no greater than 1% by mass. For producing a coloring matter containing a lower amount of the inorganic impurities, a desalting treatment may be carried out by a common method such as, for example, a method using a reverse osmosis membrane, or the like.

The ink composition of the present invention is prepared by dissolving the coloring matter represented by the formula (1) in water or an aqueous solvent (water containing a water soluble organic solvent described later); however, for example, a reaction liquid after completing the final step in synthesizing the coloring matter of the present invention, and the like can be directly used for producing the ink composition. Alternatively, a target substance may be isolated from the reaction liquid, followed by drying, e.g., spray drying, and then the substance may be processed into an ink composition. The ink composition of the present invention contains the coloring matter of the present invention in an amount of usually 0.1 to 20% by mass, more preferably 1 to 15% by mass, and still more preferably 2 to 10% by mass. The ink composition of the present invention may also contain 0 to 30% by mass of a water soluble organic solvent, and 0 to 5% by mass of an ink preparation agent, respectively.

Specific examples of the water soluble organic solvent which may be used in the present invention include C1-C4 alkanols such as methanol, ethanol, n-propanol, isopropanol, n-butanol, isobutanol, secondary butanol and tertiary butanol; carboxylic acid amides such as N,N-dimethylformamide and N,N-dimethylacetamide; lactams such as 2-pyrrolidone and N-methyl-2-pyrrolidone; cyclic ureas such as 1,3-dimethylimidazolidin-2-one and 1,3-dimethylhexahydropyrimid-2-one; ketones or keto alcohols such as acetone, methylethylketone and 2-methyl-2-hydroxypentan-4-one; cyclic ethers such as tetrahydrofuran and dioxane; mono-, oligo-, or poly-alkylene glycol or thioglycol having a C2-C6 alkylene unit such as ethylene glycol, 1,2- or 1,3-propylene glycol, 1,2- or 1,4-butylene glycol, 1,6-hexylene glycol, diethylene glycol, triethylene glycol, tetraethylene glycol, dipropylene glycol, thiodiglycol, polyethylene glycol and polypropylene glycol; triols such as glycerin and hexane-1,2,6-triol; C1-C4 alkyl ethers of a polyhydric alcohol such as ethylene glycol monomethyl ether, ethylene glycol monoethyl ether, diethylene glycol monomethyl ether, diethylene glycol monoethyl ether, triethylene glycol monomethyl ether, triethylene glycol monoethyl ether and butylcarbitol; γ-butyrolactone, dimethyl sulfoxide, and the like. These water soluble organic solvents may be used either alone, or as a mixture.

Of these, preferred are 2-pyrrolidone, N-methyl-2-pyrrolidone, mono-, di-, or tri-ethylene glycol and dipropylene glycol; and more preferred are 2-pyrrolidone, N-methyl-2-pyrrolidone, diethylene glycol and butylcarbitol.

The ink preparation agent which may be used in the ink composition of the present invention is explained below. Specific examples of the ink preparation agent include a preservative and fungicide, a pH adjusting agent, a chelating agent, a rust-preventive agent, a water soluble ultraviolet ray absorbing agent, a water soluble polymer compound, a dye solubilizer, a surfactant, and the like.

Examples of the preservative and fungicide include organic sulfur based, organic nitrogen sulfur based, organic halogen based, haloaryl sulfone based, iodopropargyl based, N-haloalkylthio based, benzothiazole based, nitrile based, pyridine based, 8-oxyquinoline based, isothiazoline based, dithiol based, pyridineoxide based, nitropropane based, organic tin based, phenol based, quaternary ammonium salt based, triazine based, thiadiazine based, anilide based, adamantane based, dithiocarbamate based, brominated indanone based, benzyl bromo acetate based, or inorganic salt based compounds, and the like.

Examples of the organic halogen based compound include sodium pentachlorophenol; examples of the pyridineoxide based compound include sodium 2-pyridinethiol-1-oxide; examples of the isothiazoline based compound include 1,2-benzisothiazolin-3-one, 2-n-octyl-4-isothiazolin-3-one, 5-chloro-2-methyl-4-isothiazolin-3-one, 5-chloro-2-methyl-4-isothiazolin-3-one magnesium chloride, 5-chloro-2-methyl-4-isothiazolin-3-one calcium chloride, 2-methyl-4-isothiazolin-3-one calcium chloride, and the like. Examples of the other preservative and fungicide include anhydrous sodium acetate, sodium sorbate, sodium benzoate, and the like.

As the pH adjusting agent, any arbitrary substance may be used as long as it can adjust the pH of the ink to fall within the range of 8.0 to 11.0 without adversely affecting the prepared ink. Examples thereof include alkanolamines such as diethanolamine and triethanolamine; hydroxides of alkali metals such as lithium hydroxide, sodium hydroxide and potassium hydroxide; ammonium hydroxide; carbonates of alkali metals such as lithium carbonate, sodium carbonate and potassium carbonate; and the like.

Examples of the chelating agent include sodium ethylenediamine tetraacetate, sodium nitrilotriacetate, sodium hydroxyethylethylenediamine triacetate, sodium diethylenetriamine pentaacetate, sodium uracil diacetate, and the like.

Examples of the rust-preventive agent include acidic sulfite, sodium thiosulfate, ammonium thioglycolate, diisopropylammonium nitrite, pentaerythritol tetranitrate, dicyclohexylammonium nitrite, and the like.

Examples of the water soluble ultraviolet ray absorbing agent include sulfonated benzophenone, sulfonated benzotriazole, and the like.

Examples of the water soluble polymer compound include polyvinyl alcohols, cellulose derivatives, polyamines, polyimines, and the like.

Examples of the dye solubilizer include urea, s-caprolactam, ethylene carbonate, and the like.

Examples of the surfactant include anionic, cationic, or nonionic well-known surfactants, and the like.

Specific examples of the anionic surfactant include alkylsulfonic acid salts, alkylcarboxylic acid salts, α-olefin sulfonic acid salts, polyoxyethylenealkyl ether acetic acid salts, N-acylamino acid and salts thereof, N-acylmethyltaurine salts, alkylsulfate polyoxyalkyl ether sulfuric acid salts, alkylsulfate polyoxyethylenealkyl ether phosphoric acid salts, rosin acid soap, castor oil sulfate ester salts, lauryl alcohol sulfate ester salts, alkylphenolic phosphate esters, alkylated phosphate esters, alkylarylsulfonic acid salts, diethyl sulfosuccinic acid salts, diethylhexyl sulfosuccinic acid salts, dioctyl sulfosuccinic acid salts, and the like.

Specific examples of the cationic surfactant include 2-vinylpyridine derivatives, poly(4-vinylpyridine) derivatives, and the like.

Specific examples of the amphoteric surfactant include lauryldimethylamino acetate betaine, 2-alkyl-N-carboxymethyl-N-hydroxyethylimidazolinium betaine, coconut oil fatty acid amidepropyldimethylamino acetate betaine, polyoctylpolyaminoethylglycine, as well as imidazoline derivatives, and the like.

Specific examples of the nonionic surfactant include: ether based surfactants such as polyoxyethylene nonylphenyl ether, polyoxyethylene octylphenyl ether, polyoxyethylene dodecyl phenyl ether, polyoxyethylene oleyl ether, polyoxyethylene lauryl ether and polyoxyethylene alkyl ether; ester based surfactants such as polyoxyethylene oleate esters, polyoxyethylene distearate esters, sorbitan laurate, sorbitan monostearate, sorbitan monooleate, sorbitan sesquioleate, polyoxyethylene monooleate and polyoxyethylene stearate; acetylene alcohol based surfactants such as 2,4,7,9-tetramethyl-5-decyne-4,7-diol, 3,6-dimethyl-4-octyne-3,6-diol and 3,5-dimethyl-1-hexyn-3-ol (for example, trade names Surfynol 104, 105, 82 and 465, and Olfine STG manufactured by Nissin Chemical Co., Ltd., etc.); polyglycol ether based surfactants (for example, Tergitol 15-S-7 manufactured by Sigma-Aldrich Co., etc.), and the like. These ink preparation agents may be used either alone or as a mixture.

In the method for producing the ink composition of the present invention, the order of dissolving each component is not particularly limited. The coloring matter may be dissolved in water or the aforementioned aqueous solvent (water that contains a water soluble organic solvent) beforehand, and thereto may be added the ink preparation agent which is then allowed to be dissolved. Alternatively, after dissolving the coloring matter in water, the water soluble organic solvent, and the ink preparation agent may be added thereto to allow them to be dissolved. Also, the ink composition may be produced by adding the water soluble organic solvent and the ink preparation agent either directly to the reaction liquid of the coloring matter, or to an aqueous solution obtained by subjecting an aqueous solution containing the coloring matter to a desalting treatment using a reverse osmosis membrane. Water employed in preparing the ink composition preferably includes impurities in an amount as low as possible, and thus water such as ion exchanged water or distilled water is preferred. Furthermore, precision filtration may be carried out to remove contaminants using a membrane filter or the like, as needed. In particular, when the ink is used as an ink jet recording ink, carrying out the precision filtration is preferred. The filter used in carrying out the precision filtration has a pore size of usually 1 μm to 0.1 μm, and preferably 0.8 μm to 0.1 μm.

The magenta ink composition containing the coloring matter of the present invention is suited for use in a printing, copying, marking, writing, drawing, stamping, or recording method, and particularly in ink jet recording. When the ink composition of the present invention is used for recording, particularly for ink jet recording, high quality magenta recorded matters having favorable resistance to water, sunlight, ozone, and friction are obtained. In addition, by further blending a well-known coloring matter such as yellow or magenta to the coloring matter of the present invention as needed in the range without deteriorating the effects achieved by the ink composition and the like of the present invention, color tone of magenta can be also adjusted to a favorite color tone such as orange, reddish, or the like. Moreover, the coloring matter of the present invention may be used as a coloring matter for adjusting a color to be contained in other color, particularly in a blended black ink.

The colored body of the present invention is a substance which was colored with the ink composition or the coloring matter of the present invention. The object to be colored is not particularly limited, and, for example, a paper, a fiber or cloth (cellulose, nylon, wool, etc.), leather, a substrate for color filters, and the like are exemplified. The coloring method may include, for example, printing methods such as a dip dyeing method, a textile printing method and a screen printing, as well as recording methods with an ink jet printer, and the like, but the recording method with an ink jet printer is preferred.

Record-receiving materials (media) to which the ink jet recording method of the present invention is applicable include, for example, communication sheets such as paper and films, as well as fibers, leather and the like, and communication sheets are preferred. The communication sheet is preferably a material subjected to a surface treatment, specifically, paper, films etc., as the base material, having an ink receiving layer provided thereon. The ink-receiving layer is provided by, for example, impregnating or coating a cationic polymer into or onto the aforementioned base material; coating a porous white inorganic substance that can absorb a coloring matter in an ink such as porous silica, alumina sol or special ceramics onto the surface of the aforementioned base material together with a hydrophilic polymer such as polyvinyl alcohol or polyvinylpyrrolidone; or the like. Such sheets provided with an ink receiving layer are generally referred to as ink jet exclusive paper (film) or glossy paper (film), and, for example, trade name Pictorico, manufactured by Asahi Glass Co., Ltd.; trade names: Professional Photo Paper, Super Photo Paper, Matte Photo Paper, and Photo paper • (Glossy), manufactured by Canon, Inc.; trade names: Photo Paper <Glossy>, Photo Matte Paper, and Superfine Glossy Exclusive Film, manufactured by Epson Corporation; trade names: Advanced Photo Paper, Premium Plus Photo Paper, Premium Gloss Film, and Photo Paper, manufactured by Hewlett-Packard Japan, Ltd.; trade name: Photolike QP, manufactured by Konica Corporation; and the like are included. As a matter of course, plain paper can be also used.

In these regards, images recorded on a record-receiving material coated with porous white inorganic substance on the surface thereof are known to be significantly discolored and faded due to ozone gas in particular; however, the aqueous magenta ink composition of the present invention exhibits particularly favorable effects in recording on such record-receiving materials since the composition is superior in gas resistance.

Examples of the porous white inorganic substance include calcium carbonate, kaolin, talc, clay, diatomaceous earth, synthetic amorphous silica, aluminum silicate, magnesium silicate, calcium silicate, aluminum hydroxide, alumina, lithopone, zeolite, barium sulfate, calcium sulfate, titanium dioxide, zinc sulfide, zinc carbonate, and the like.

For recording on a record-receiving material with the ink jet recording method of the present invention, for example, a vessel including the ink composition is set at a specified position of an ink jet printer, and the recording may be executed by a conventional method on the record-receiving material. In the ink jet recording method of the present invention, a green ink composition, an orange ink composition, a blue (or violet) ink composition, the magenta ink composition of the present invention, and/or a black ink composition etc., if necessary, may be used in combination, in addition to a well-known yellow ink composition and a cyan ink composition. The ink composition of each color is injected into each of vessels, and these vessels may be set (attached) at a specified position of the ink jet printer, similarly to the vessel including the aqueous magenta ink composition of the present invention, and then used. As the ink jet printer, for example, a printer of a piezo system utilizing mechanical vibration; a printer of a bubble jet (registered trademark) system utilizing bubbles generated by heating; and the like may be adopted.

The ink composition of the present invention is an aqueous ink composition that provides a recorded image having a brilliant magenta color and particularly having a highly brilliant hue on ink jet glossy paper, and also the recorded image has superior fastness properties. In addition, the ink composition has a high level of safety in humans.

The ink composition of the present invention is precluded from precipitation and separation during storage. Also, when the ink of the present invention is used in ink jet recording, clogging of the injector (ink head) can be avoided. The ink composition of the present invention does not cause alteration of physical properties even in the case of: use under certain recycling for a comparatively long time with a continuous ink jet printer; intermittent use with an on-demand ink jet printer; or the like.

### EXAMPLES

Hereinafter, the present invention is specifically described by way of Examples. In the specification, the expressions “part” and “%” are on the basis of the mass unless otherwise stated particularly. Furthermore, each operation of synthetic reaction, crystallization and the like was carried out under stirring unless otherwise stated particularly, and the reaction temperature disclosed is a measured value of the temperature in the reaction system. It should be noted that each coloring matter of the present invention synthesized in Examples was subjected to measurement of λmax (wavelength of maximum absorption) in water, and thus obtained measurement value is shown.

### Example 1

### Step 1

Into 75.0 parts of orthodichlorobenzene were added 23.6 parts of a compound represented by the following formula (13) obtained by a well-known method, 0.75 parts of sodium carbonate and 36.0 parts of ethyl benzoylacetate serially while stirring, and the temperature of the mixture was elevated, followed by allowing to react at a temperature of 170 to 175° C. for 3 hrs. After completing the reaction, the reaction liquid was cooled, and thereto were added 150 parts of methanol at 30° C. After the mixture was stirred for 30 min, precipitated solid was separated by filtration. Thus obtained solid was washed with 200 parts of methanol, followed by washing with water. The solid was dried to obtain 28.8 parts of a compound represented by the following formula (14) as red solid.

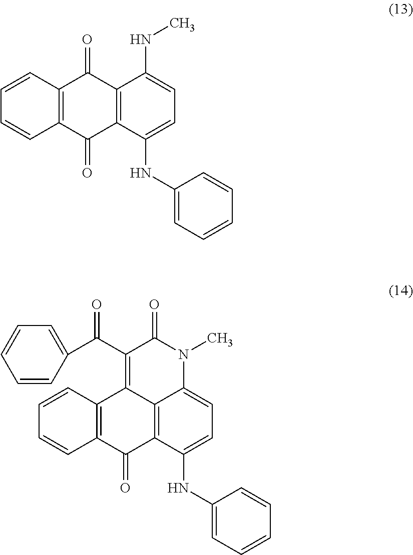

### step 2

At room temperature, 14.0 parts of the compound represented by the above formula (14) were added to 116.5 parts of chlorosulfonic acid such that the temperature did not exceed 40° C. Thereafter, the temperature was elevated to 100° C., and the mixture was stirred for 4 hrs. The reaction liquid was cooled to 70° C., and 53.5 parts of thionyl chloride were added dropwise over 30 min at the same temperature. Thereafter, the reaction was allowed at 70° C. for 3 hrs. The reaction liquid left to cool down to room temperature was charged into 500 parts of ice water, and then 20 parts of aqueous hydrogen peroxide were further added thereto. During this step, ice was added appropriately, whereby the liquid temperature was maintained at no higher than 10° C. Thus precipitated reddish orange solid was separated by filtration to obtain 90.0 parts of a wet cake of a compound represented by the following formula (15). It should be noted that the compound represented by the following formula (15) is a compound represented by the above formula (5) in which Q is a chlorine atom.

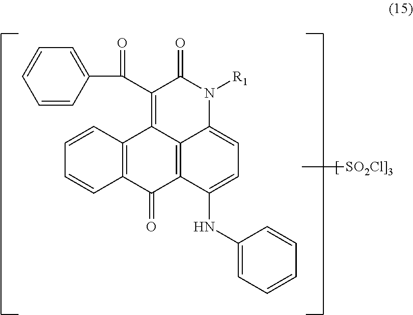

### Step 3

Into 300 parts of ice water were added 45.0 parts of the compound represented by the above formula (15), and the mixture was stirred for 10 min. Thereafter, 2.3 parts of glycine were added thereto, and the reaction was allowed for 30 min while maintaining a pH of 9.0 after adding sodium hydroxide at 20° C. The temperature of the reaction liquid was elevated to 50° C., and to the liquid were added 110.0 parts of ammonium chloride. Thus precipitated solid was separated by filtration. The solid was washed with 100 parts of a 22% aqueous ammonium chloride solution, and desalted with a mixed solution of methanol and isopropyl alcohol, followed by drying to obtain as red solid 6.0 parts of an ammonium salt of the coloring matter of the present invention represented by the following formula (16), which is a coloring matter represented by the above formula (4) in which h is 1.8; j is 1.2; and X_{d} is carboxymethylamino. λmax: 523 nm.

Results of HPLC analysis:

| Number of substitution with —SO₂X_{d} | HPLC area ratio (%) |
|---|---|
| 0 | 3.4 |
| 1 | 23.3 |
| 2 | 42.9 |
| 3 | 15.7 |

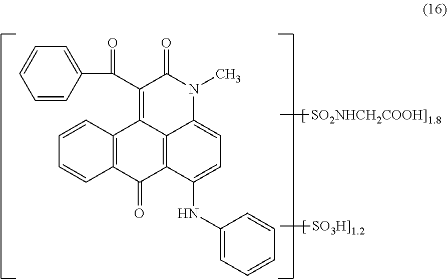

### Example 2

Into 300 parts of ice water were added 45.0 parts of the compound represented by the formula (15) obtained similarly to (Step 1) to (Step 3) of Example 1, and the mixture was stirred for 10 min. Thereafter, 6.8 parts of glycine were added thereto, and the reaction was allowed for 30 min while maintaining a pH of 9.0 after adding sodium hydroxide at 20° C. The temperature of the reaction liquid was elevated to 50° C., and to the liquid were added 110.0 parts of ammonium chloride. The mixture was stirred, and thus precipitated solid was separated by filtration. The solid was washed with 100 parts of a 22% aqueous ammonium chloride solution, and desalted with a mixed solution of methanol and isopropyl alcohol, followed by drying to obtain as red solid 6.0 parts of an ammonium salt of the coloring matter of the present invention represented by the following formula (17), which is a coloring matter represented by the above formula (4) in which h is 2.2; j is 0.8; and X_{d} is carboxymethylamino. λmax: 535 nm.

Results of HPLC analysis:

| Number of substitution with —SO₂X_{d} | HPLC area ratio (%) |
|---|---|
| 0 | 0.8 |
| 1 | 12.3 |
| 2 | 42.2 |
| 3 | 35.2 |

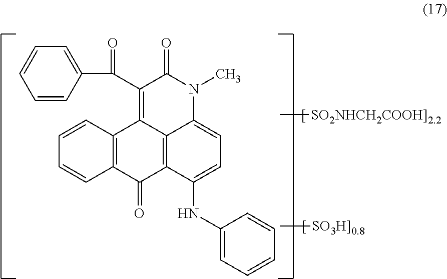

### Example 3

Into 300 parts of ice water were added 45.0 parts of the compound represented by the formula (15) obtained similarly to (Step 1) to (Step 3) of Example 1, and the mixture was stirred for 10 min. Thereafter, 12.3 parts of anthranilic acid were added thereto, and the reaction was allowed for 30 min at a pH of 9.0 at 20° C. The temperature of the reaction liquid was elevated to 50° C., and to the liquid were added 45.0 parts of ammonium chloride. The mixture was stirred, and thus precipitated solid was separated by filtration. The solid was sequentially washed with 100 parts of a 19% ammonium chloride aqueous solution and 3% hydrochloric acid, and dried to obtain as red solid 14.0 parts of an ammonium salt of the coloring matter of the present invention represented by the following formula (18), which is a coloring matter represented by the above formula (4) in which h is 2.3; j is 0.7; and X_{d} is 2-carboxyanilino. λmax: 528 nm.

Results of HPLC analysis:

| Number of substitution with —SO₂X_{d} | HPLC area ratio (%) |
|---|---|
| 0 | 0.7 |
| 1 | 10.6 |
| 2 | 40.6 |
| 3 | 33.8 |

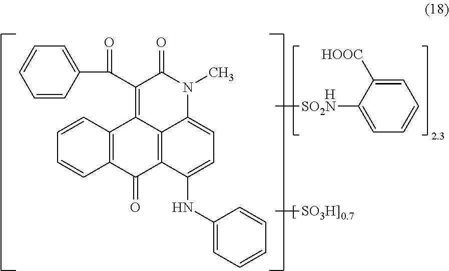

### Example 4

Into 300 parts of ice water were added 45.0 parts of the compound represented by the formula (15) obtained similarly to (Step 1) to (Step 3) of Example 1, and the mixture was stirred for 10 min. Thereafter, 12.0 parts of iminodiacetic acid were added thereto, and the reaction was allowed for 30 min while maintaining a pH of 9.0 after adding sodium hydroxide at 20° C. The pH of this reaction liquid was adjusted to 0.5 with conc. hydrochloric acid, and the deposited oily matter was dissolved in aqueous ammonia. Thereafter, the pH of the solution was adjusted again to 0.5 with conc. hydrochloric acid, and the obtained oily matter was separated by decantation, to which water was added. Following decantation carried out again, the oily matter was dried to obtain as red solid 1.63 parts of an ammonium salt of the coloring matter of the present invention represented by the following formula (19), which is a coloring matter represented by the above formula (4) in which h is 2.3; j is 0.7; and X_{d} is bis(carboxymethyl)amino. λmax: 527 nm.

Results of HPLC analysis:

| Number of substitution with —SO₂X_{d} | HPLC area ratio (%) |
|---|---|
| 0 | 0.7 |
| 1 | 11.6 |
| 2 | 39.0 |
| 3 | 34.5 |

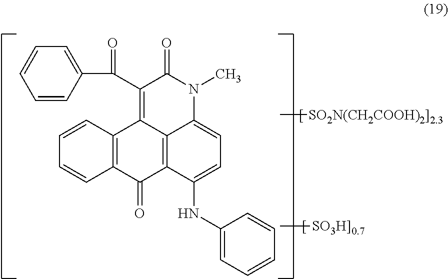

### Example 5

Into 300 parts of ice water were added 45.0 parts of the compound represented by the formula (15) obtained similarly to (Step 1) to (Step 3) of Example 1, and the mixture was stirred for 10 min. Thereafter, 11.6 parts of isonipecotic acid were added thereto, and the reaction was allowed for 30 min while the pH was maintained at 9.0 after adding sodium hydroxide at 20° C. The temperature of the reaction liquid was elevated to 50° C., and to the liquid were added 9.0 parts of ammonium chloride. The mixture was stirred, and thus precipitated solid was separated by filtration. The solid was washed sequentially with 100 parts of a 7% aqueous ammonium chloride solution and methanol, followed by drying to obtain as red solid 11.4 parts of an ammonium salt of the coloring matter of the present invention represented by the following formula (20), which is a coloring matter represented by the above formula (4) in which h is 2.4; j is 0.6; and X_{d} is 4-carboxypiperidin-1-yl (1-isonipecotinyl). λmax: 524 nm.

Results of HPLC analysis:

| Number of substitution with —SO₂X_{d} | HPLC area ratio (%) |
|---|---|
| 0 | 0.4 |
| 1 | 7.6 |
| 2 | 38.3 |
| 3 | 43.4 |

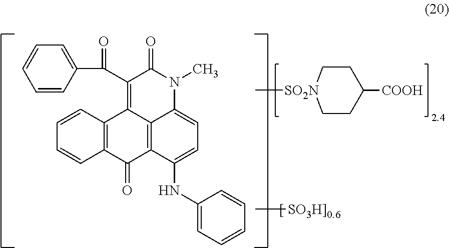

### Examples 6 to 10

### (A) Preparation of Ink

Using the coloring matter obtained in Example 1, an ink composition constituted as shown in Table 2 below was prepared, which was filtered through a 0.45 μm membrane filter to provide an aqueous ink jet ink for evaluation. Water employed for preparing the ink was ion exchanged water. The pH of the ink composition was adjusted with a 18% aqueous ammonia solution to give a pH of 8 to 10, and water was further added thereto to make the total amount of 100 parts. Preparation of the ink for evaluation using the coloring matter obtained in Example 1 is designated as Example 6. Similarly, preparations of the inks for evaluation using the coloring matters obtained in Examples 2 to 5 are designated as Examples 7 to 10, respectively.

TABLE 2

| | |
|---|---|
| compound obtained in Example 1 | 6.0 parts |
| glycerin | 5.0 parts |
| urea | 5.0 parts |
| N-methyl-2-pyrrolidone | 4.0 parts |
| IPA (Isopropyl alcohol) | 3.0 parts |
| butylcarbitol | 2.0 parts |
| surfactant (trade name: Surfynol 104 PG50, | 0.1 parts |
| manufactured by Nissin Chemical Co., Ltd.) | |
| 18% aqueous ammonia solution + water | 74.9 parts |
| total | 100.0 parts |

### Comparative Example 1

An ink for comparison was prepared in a similar manner to Example 6 except that a coloring matter represented by the following formula (21), which had been obtained from a wet cake synthesized in accordance with Example 1, (1) to (3) in PCT International Publication No. 2008/018495, by drying at 80° C. overnight was used in place of the coloring matter of Example 1 described above. This preparation of the ink for comparison is designated as Comparative Example 1.

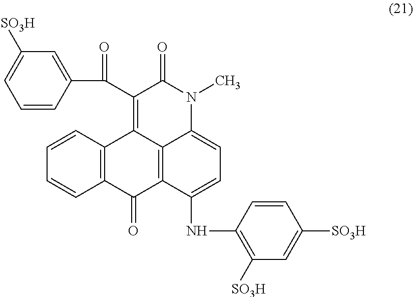

### (B) Ink Jet Recording

Using an ink jet printer (manufactured by Canon, Inc., trade name: Pixus iP4100), ink jet recording was carried out on three types of glossy papers having an ink-receiving layer that contains a porous white inorganic substance (trade name: Professional Photo Paper PR-101; and trade name: Photo Paper Gloss Gold GL-101, manufactured by Canon, Inc., and trade name: Advanced Photo Paper, manufactured by Hewlett-Packard Japan, Ltd.). These three types of glossy papers were named as glossy paper 1, glossy paper 2, and glossy paper 3, respectively. Upon ink jet recording, an image pattern was produced such that several-step gradation of the printed density was obtained, whereby a printed matter for evaluation test was produced, which was used as a test piece for carrying out an ozone gas resistance test described below.

### (C) Ozone Gas Resistance Test of Recorded Image

Each test piece obtained according to “(B) Ink Jet Recording” described above was left to stand under a condition of: an ozone concentration of 10 ppm; a temperature of 24° C.; and a humidity of 60% RH, using an Ozone Weather Meter (manufactured by Suga Test Instruments Co., Ltd.) for 24 hrs. Thus, the reflected density was determined by colorimetry before and after the test at a gradation portion having a reflected density (D value) that is most approximate to 1.0 before the test. The reflected density was determined using a colorimetric system (manufactured by Gretag Macbeth Co., trade name: Sectro Eye). It should be noted that the evaluation was carried out by comparing the residual ratio of the coloring matter represented by the following formula. Larger value of the residual ratio of the coloring matter indicates more superior ozone gas resistance. The results are shown in the following Table 3.

Residual ratio of the coloring matter=(Reflected density after test/Reflected density before test)×100(%)

TABLE 3
Residual ratio of the coloring matter

| | glossy paper 1 | glossy paper 2 | glossy paper 3 |
|---|---|---|---|
| Example 6 | 88.7 | 86.4 | 92.0 |
| Example 7 | 90.2 | 88.7 | 92.6 |
| Example 8 | 86.6 | 87.5 | 90.3 |
| Example 9 | 90.5 | 89.6 | 92.2 |
| Example 10 | 87.9 | 90.7 | 95.1 |
| Comparative | 84.1 | 85.6 | 88.3 |
| Example 1 | | | |

As is clear from Table 3, it is proven that Examples 6 to 10 resulted in higher residual ratios on all glossy papers than Comparative Example 1, suggesting very favorable ozone gas resistance.

As described above, it is evident that the recorded image obtained with the ink composition prepared using the coloring matter of the present invention is excellent in ozone gas resistance. Therefore, the magenta ink composition and the magenta coloring matter of the present invention are extremely useful for ink jet recording.

1. An ink composition comprising as a coloring matter at least one anthrapyridone coloring matter represented by the following formula (1) or a salt thereof 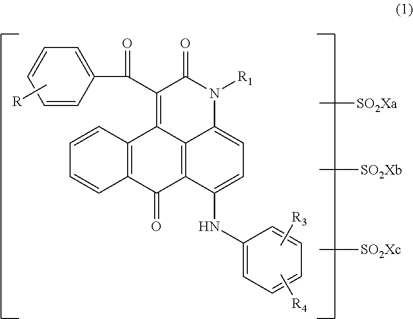
wherein,
Xₐ to X_{c} each independently represent an anilino group substituted with a carboxy group, a mono- or di-alkylamino group substituted with a carboxy group, or a hydroxy group, and at least one of Xₐ to X_{c} represents a group other than a hydroxy group;
R represents a hydrogen atom, a sulfo group, a carboxy group, an alkoxy group, an alkylthio group, a carbamoyl group, a cyano group, an alkyl group, an anilino group, a phenoxy group, an amino group, a hydroxy group, or a mercapto group;
R₁ represents a hydrogen atom, an alkyl group, a hydroxyalkyl group, a phenyl group, a mono- or di-alkylaminoalkyl group, or a cyano alkyl group; and
R₃ and R₄ each independently represent a hydrogen atom or an alkyl group.

2. The ink composition according to claim 1, wherein the anthrapyridone coloring matter represented by the above formula (1) or a salt thereof is an anthrapyridone coloring matter represented by the following formula (2) or a salt thereof 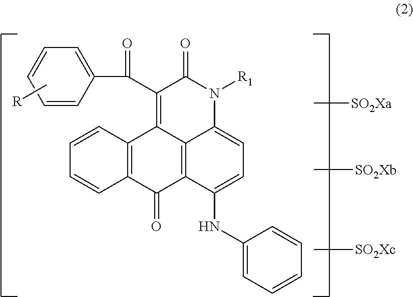
wherein, Xₐ to X_{c}, R, and R₁ are as defined in the formula (1).

3. The ink composition according to claim 1, wherein the anthrapyridone coloring matter represented by the above formula (1) or a salt thereof is an anthrapyridone coloring matter represented by the following formula (3) or a salt thereof 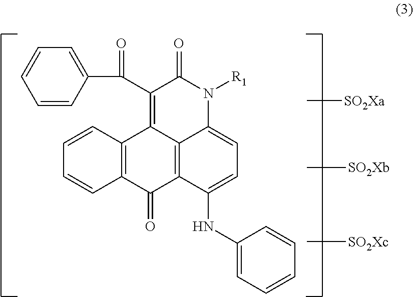
wherein, Xₐ to X_{c}, and R₁ are as defined in the formula (1).

4. The ink composition according to claim 1, wherein the anthrapyridone coloring matter represented by the above formula (1) or a salt thereof is an anthrapyridone coloring matter represented by the following formula (4) or a salt thereof 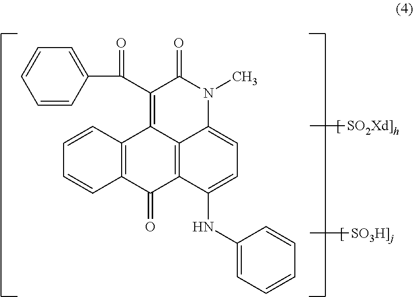
wherein,
X_{d} represents an anilino group substituted with a carboxy group, or a mono- or di-alkylamino group substituted with a carboxy group; h and j are both an average value; h is from 1.6 to 2.5; j is from 0.5 to 1.4; and the sum of h and j is 3.0.

5. The ink composition according to claim 1, wherein the anthrapyridone coloring matter represented by the above formula (1) or a salt thereof is a coloring matter or a salt thereof obtained by reacting a compound represented by the following formula (5) with at least one amine selected from the group consisting of aniline substituted with a carboxy group, and a mono- or di-alkylamine substituted with a carboxy group 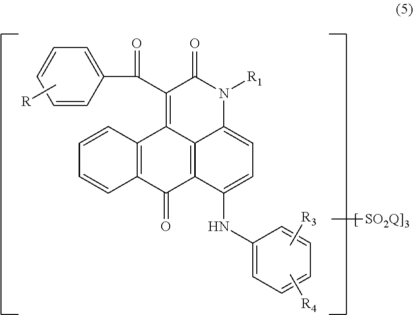
wherein, Q represents a halogen atom; and R, R₁, R₃, and R₄ are as defined in the formula (1).

6. The ink composition according to claim 1 comprising water and a water soluble organic solvent.

7. The ink composition according to claim 1, wherein the content of inorganic impurities in the total mass of the anthrapyridone coloring matter represented by the above formula (1) or a salt thereof contained in the ink composition as a coloring matter is no greater than 1% by mass.

8. The ink composition according to claim 1, wherein the content of the anthrapyridone coloring matter represented by the above formula (1) or a salt thereof contained in the ink composition as a coloring matter is 0.1 to 20% by mass relative to the total mass of the ink composition.

9. The ink composition according to claim 1, which is for use in ink jet recording.

10. An ink jet recording method comprising recording on a record-receiving material using as an ink the ink composition according to claim 1 by discharging ink droplets of the ink in response to recording signals.

11. The ink jet recording method according to claim 10, wherein the record-receiving material is a communication sheet.

12. The ink jet recording method according to claim 11, wherein the communication sheet is a sheet having an ink-receiving layer comprising a porous white inorganic substance.

13. A colored body which was colored with the ink composition according to claim 1.

14. The colored body according to claim 13, wherein the coloring is carried out with an ink jet printer.

15. An ink jet printer equipped with a vessel containing the ink composition according to claim 1.

16. An anthrapyridone coloring matter represented by the following formula (4) or a salt thereof 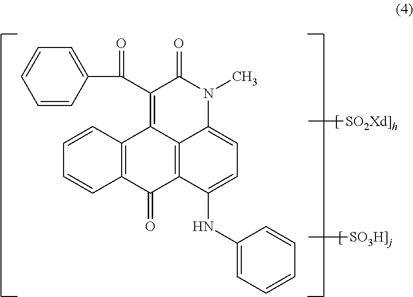
wherein,
X_{d} represents an anilino group substituted with a carboxy group, or a mono- or di-alkylamino group substituted with a carboxy group; h and j are both an average value; h is from 1.6 to 2.5; j is from 0.5 to 1.4; and the sum of h and j is 3.0.
